(19) United States
(12) Patent Application Publication
(10) Pub. No.: US 2026/0345153 A1
(43) Pub. Date: Nov. 5, 2026

(54) Hooked suture thread with cannulated needle and stylet for precision surgery

(71) Applicants: Benedict de St. Amatus, Forest Glen (AU); Alexander de St. Amatus, Forest Glen (AU)

(72) Inventors: Benedict de St. Amatus, Forest Glen (AU); Alexander de St. Amatus, Forest Glen (AU)

(21) Appl. No.: 19/197,384

(22) Filed: May 2, 2025

(51) Int. Cl. A61B 17/06; A61B 17/04; A61B 17/062

(52) U.S. Cl. CPC A61B 17/06166; A61B 17/0493; A61B 17/06066; A61B 17/0625; A61B 2017/06052; A61B 2017/061; A61B 2017/0618

(57) ABSTRACT

The present invention introduces a hooked suture thread system designed for precision in minimally invasive surgical procedures. The suture thread features a hook at one end and a straight end with apertures along its length. The hook terminates in a blunt end, allowing for precise positioning at any desired depth and angle within the subdermal or hypodermal layers of skin tissue or other body tissue. The system includes a stylet encasing the suture thread in a longitudinal channel, facilitating controlled forward, backward, and rotational movements within a cannulated needle. The needle, equipped with a bevel for tissue penetration and interlocking handles, ensures stable positioning and easy manipulation. The suture's flexible material ensures secure tissue approximation while minimising trauma. The invention also provides a straightforward removal process, enhancing both the efficiency and outcomes of surgical procedures.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### FIELD OF THE INVENTION

The present invention relates to devices used in medical procedures, specifically those involved in plastic and cosmetic surgical operations and tissue repair. More particularly, the invention pertains to the field of minimally invasive surgical techniques for reconstructive procedures requiring precise tissue repair and the restoration of normal function and appearance, as well as aesthetic procedures aimed at enhancing appearance, achieving minimal scarring, and precise tissue approximation with reduced tissue damage, using suture threads and associated instruments.

### DESCRIPTION OF RELATED ART

Sutures are medical devices used to hold body tissues together after an injury or surgery. They are essentially threads or strands that surgeons use to stitch wounds or surgical incisions to promote healing and prevent infection. Sutures can be made from various materials, including absorbable substances that the body can naturally break down over time, and non-absorbable materials that may need to be removed once the wound has sufficiently healed. The design of sutures ranges from simple straight threads to more complex knotless anchoring barbed, button and hooked versions, which can offer enhanced tissue approximation and stability without the need for knots. In minimally invasive surgical procedures, specialised instruments like cannulated needles and stylets are often employed alongside sutures to allow for precise and controlled placement, reducing tissue trauma and improving cosmetic outcomes.

The knotless anchoring sutures come in various designs, each tailored to provide enhanced stability and effectiveness for specific surgical applications. In particular:

The barbed sutures represent a significant advancement in suture technology, incorporating small, evenly spaced barbs along the length of the suture thread. These barbs engage with the surrounding tissue as the suture is drawn through, effectively anchoring the suture without the need for knots. Unidirectional barbs face one direction and are ideal for procedures where the tissue needs to be drawn together in a single direction, providing a secure hold that prevents the suture from slipping backward. Bidirectional barbs, facing in both directions, offer even greater stability by anchoring the suture at multiple points along its path, which is particularly beneficial in complex, wound closures where maintaining precise tissue alignment is critical. This technology reduces the risk of wound dehiscence (reopening) and minimises tissue trauma, as the barbs allow for secure fixation with fewer passes through the tissue, thus reducing the overall surgical time and enhancing healing outcomes.

The anchoring buttons are small, disk-like structures that are used in conjunction with sutures to provide enhanced stability and tension distribution across the wound site. These buttons are placed on the tissue surface and serve as anchor points for the suture thread, preventing the suture from pulling through the tissue. By distributing the tension more evenly, anchoring buttons help maintain precise tissue alignment and reduce the risk of tissue tearing or suture migration. This is especially important in procedures where maintaining the exact positioning of tissue layers is critical, such as in reconstructive surgeries or in areas subjected to significant mechanical stress. Anchoring buttons also provide additional support in high-tension areas, ensuring that the wound edges remain approximated and stable throughout the healing process. Their use can lead to improved surgical outcomes, reduced scarring, and enhanced patient recovery by promoting more efficient and secure wound closure.

The hooked sutures are designed with small hooks or anchoring elements strategically placed along the length of the suture thread. These hooks engage with the tissue as the suture is advanced, providing a secure and stable hold that prevents the suture from slipping or migrating. The hooks are crafted to minimise tissue damage while offering strong anchoring points, making them particularly useful in areas where tissue movement is high or where a strong initial hold is required. This is especially important in reconstructive surgeries, where precise tissue alignment and stability are critical for functional and aesthetic outcomes. The design of hooked sutures allows for better control and placement during the suturing process, leading to improved wound healing and reduced scarring. Additionally, the hooks provide an extra layer of security, ensuring that the suture remains in place throughout the healing process, even in dynamic and high-stress environments.

Prior art techniques in the realm of surgical suturing, particularly with the use of hooked suture threads combined with cannulated needles and stylets, present several notable disadvantages. Traditional sutures, while effective for wound closure, often result in significant tissue trauma due to the repetitive puncturing required for knotting and securing the sutures. This can lead to increased healing times and greater scarring, which are particularly undesirable in cosmetic and reconstructive surgeries. Additionally, conventional sutures may lack the precision needed for optimal tissue approximation, especially in minimally invasive procedures where accurate placement is crucial. Existing technologies such as barbed sutures, anchoring buttons, and hooked sutures, although improvements over basic suturing methods, still face limitations. Barbed sutures, for instance, can cause tissue irritation and may not offer the necessary flexibility for complex tissue alignments. Anchoring buttons, while providing stability, can add bulk and may not be suitable for all surgical applications. Hooked sutures provide a secure hold but can be challenging to remove, potentially causing further tissue damage during extraction. These limitations highlight the need for an advanced suturing system that offers precise placement, minimal tissue trauma, and ease of removal, which the present invention aims to address.

### OBJECT OF THE INVENTION

It is an object of the present invention to overcome, or at least substantially ameliorate, the disadvantages and shortcomings of the prior art related to hooked suture threads with cannulated needles and stylets for precision surgery.

In particular, the object of the present invention is to provide hooked suture threads that can be precisely positioned at any desirable depth and angle within a tissue, ensuring optimal placement and effectiveness during surgical procedures.

Another object of the present invention is to facilitate the removal of the hooked suture thread from the tissue when necessary, providing a straightforward and efficient method for suture extraction without causing undue trauma to the surrounding tissue.

Other objects and advantages of the present invention will become apparent from the following description, taken in connection with the accompanying drawings, wherein, by way of illustration and example, embodiments of the present invention are disclosed.

### SUMMARY OF THE INVENTION

According to the present invention, the hooked suture thread comprises a suture thread having a first end formed into at least one hook and a second end remaining straight. The suture thread further includes at least one aperture along its length. The hook at the first end terminates in a blunt end, the blunt end being positioned and applied at a precise depth and angle within the subdermal or hypodermal layers of skin tissue or other body tissue. The straight end of the hooked suture thread is then pulled to apply pressure to the tissue at the blunt end point. This straight end is subsequently stitched through its aperture to another tissue point, thereby creating tension between the two distant tissue points.

The material of the hook is elastic, allowing for a narrower distance between its blunt end and the suture thread when pressed, and a wider distance when not pressed. The elasticity and flexibility of the hook material prevent the hook from unbending. The material of the suture thread is of sufficient strength to provide tension between the point on the tissue where the blunt end of the hook is positioned and applied, and the point where the straight end of the suture thread is stitched to the tissue. Additionally, the material possesses sufficient flexibility and low friction to allow for unobstructed manoeuvrability during placement.

The suture thread is encased in a longitudinal channel within a stylet. The stylet comprises a cylindrical body with a diameter that fits within a cannulated needle, permitting unobstructed forward, backward, and rotational motion within the needle. It includes a longitudinal channel that accommodates the suture thread. Housings at the stylet tip are matched to the hook and blunt end of the suture thread. These housings function as tillers, allowing for the rotation of the suture thread, the hook, and the blunt end, thereby enabling the positioning of the blunt end at any desired angle. A retaining cap on the stylet tip locks the blunt end in place. An oval-shaped handle provides rotation control and facilitates pushing the stylet and the hooked suture thread through the cannulated needle. Additionally, the handle features a hand guard to prevent slipping and provide leverage. The stylet handle also contains a longitudinal channel that facilitates suture thread release, with sigmoid curvatures designed to lock and immobilise the suture thread when needed.

The cannulated needle for providing a conduit for guiding and positioning the stylet and the hooked suture thread comprises a cylindrical cannulated needle. It includes a needle handle that encases the cannulated needle. The cannulated needle is equipped with a bevel for tissue penetration. An oval-shaped needle handle is provided for rotation control. Additionally, the needle handle features a hand guard to prevent slipping and provide leverage. The back end surface of the needle handle is equipped with interlocking features comprising protrusions and recesses. These interlocking features match opposite-shaped elements on the stylet handle, allowing for stable locking of the needle handle to the stylet handle. The needle handle and its interlocking features are equipped with a sleeve bearing that allows rotational movement of the interlocking features of the needle handle and those of the stylet handle locked to the needle handle, thereby enabling precise positioning of the stylet handle, the stylet and its tip, the hooked suture thread, and its blunt end at any angle relative to the axis of the cannulated needle.

The stages of application of the hooked suture thread by a surgeon involve the percutaneous insertion of the cannulated needle through the skin to access the underlying tissue. The suture thread is then inserted with the assistance of the stylet into the cannulated needle, and both the suture thread and stylet are pushed towards the tip of the cannulated needle until the hook and blunt end of the suture thread reach the tip of the cannulated needle. The surgeon rotates the stylet handle, the stylet and its tip, the hooked suture thread and its blunt end, and positions the blunt end at any desired angle relative to the axis of the cannulated needle. At this point, the interlocking features of the needle handle and stylet handle lock. The retaining cap unlocks the hook and blunt end of the suture thread from the tip of the stylet by pulling down and releasing the straight end of the suture thread from the longitudinal channel of the stylet and stylet handle, securing the blunt end of the suture thread to the desired position within the tissue. The stylet is retracted from the cannulated needle, and the cannulated needle is extracted from the tissue and skin. Subsequently, the straight end of the hooked suture thread is pulled to apply pressure to the tissue at the blunt end point, and the straight end is stitched through its aperture to another tissue point, thereby creating tension between the two distant tissue points.

The hook facilitates the removal of the suture thread and blunt end when necessary. The removal procedure comprises making an incision at the dermis near the position of the hook. The tissue is then dissected from the straight end of the suture thread. The hook is interlocked with a surgical hook retractor, and subsequently, the suture thread and the blunt end are pulled out.

### BRIEF DESCRIPTION OF THE DRAWING

By way of illustration only, and with proportions of the parts not being to scale, an embodiment of the invention is described more fully hereinafter with reference to the accompanying drawings, in which:

FIG. 1 depicts a side view of the suture thread, along with views of the suture thread assembled with the cannulated needle and its handle, and the stylet and its handle.

FIG. 2 shows views of the tips of the cannulated needle and the stylet, and a radial cross-sectional view demonstrating the suture thread within the stylet and the cannulated needle.

FIG. 3 provides views of the cannulated needle and its handle.

FIG. 4 presents views of the stylet and its handle.

FIG. 5 depicts the stages of applying the suture thread.

### DETAILED DESCRIPTION OF THE INVENTION

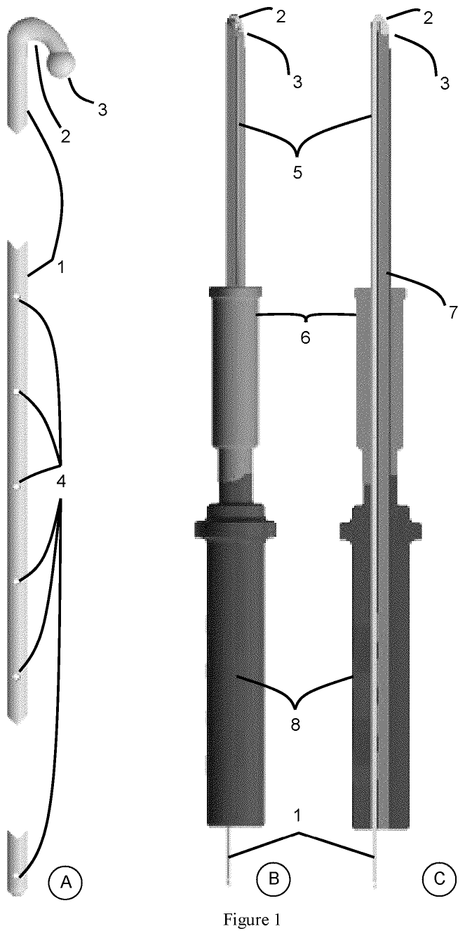

View A of FIG. 1 illustrates a suture thread (1), which is designed to provide tension between two distant points of the subdermal or the hypodermal layers of the skin's tissue or other body tissues, ensuring secure and precise post-operational tissue's approximation. One end of the suture thread (1) is formed into at least one hook (2), while the other end remains straight. The hook (2) terminates in a blunt end (3). In this embodiment, the suture thread (1) is equipped with a plurality of apertures (4) along its length, which serve to facilitate the stitching the straight end of the suture thread (1) to the desired position on the tissue.

The blunt end (3) reduces pressure on the tissue when tension is applied to the suture thread (1) and distributes the pressure evenly, thereby protecting the tissue from damage.

The material chosen for the suture thread (1) is of sufficient strength to provide tension between the point on the tissue where the blunt end (3) of the hook (2) is positioned and applied and the point where the straight end of the suture thread (1) is stitched to the tissue. Additionally, the material is selected to allow for unobstructed manoeuvrability within the tissues during placement.

The material of the suture thread (1) is of sufficient strength to provide tension between the point on the tissue where the blunt end (3) of the hook (2) is positioned and applied, and the point where the straight end of the suture thread (1) is stitched to the tissue. Additionally, the material possesses sufficient flexibility and low friction to allow for easy manoeuvrability during placement.

The material from which the hook (2) is made is sufficiently elastic to enable a narrower distance between its blunt end (3) and the suture thread (1) when pressed, and a wider distance when not pressed. The elasticity and flexibility of the hook (2) material are selected to prevent the hook from unbending.

View B of FIG. 1 shows a side view of the suture thread (1), the hook (2), and the blunt end (3) as they are positioned inside a cylindrical cannulated needle (5). The function of the cannulated needle (5) is to provide a conduit for precisely guiding and placing the suture thread (1) with minimally invasive access and minimal tissue damage. The cannulated needle (5) is equipped with a needle handle (6). A stylet (7) with a stylet handle (8) is employed to push the suture thread (1) through the cannulated needle (5) to the precise location within the tissue.

View C of FIG. 1 depicts a longitudinal cross-section of the suture thread (1), the hook (2), the blunt end (3), the cannulated needle (5), the needle handle (6), the stylet (7), and the stylet handle (8).

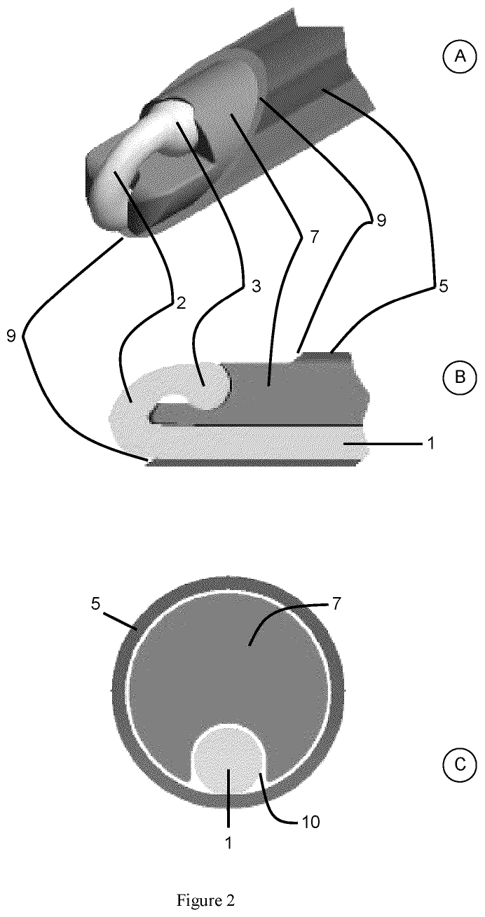

View A of FIG. 2 illustrates an axonometric view of the tip of the cannulated needle (5), wherein the cannulated needle (5) features a bevel (9) at its sharp end, designed for efficient penetration through the tissue.

View B of FIG. 2 depicts a longitudinal cross-section of the tip of the cannulated needle (5).

Views A and B of FIG. 2 also show the suture thread (1), the hook (2), the blunt end (3), and the stylet (7) as positioned at the tip of the cannulated needle (5).

View C of FIG. 2 depicts a radial cross-section of the cannulated needle (5), showing the suture thread (1) and the stylet (7) inside, wherein the suture thread (1) is positioned within a longitudinal channel (10) of the stylet (7).

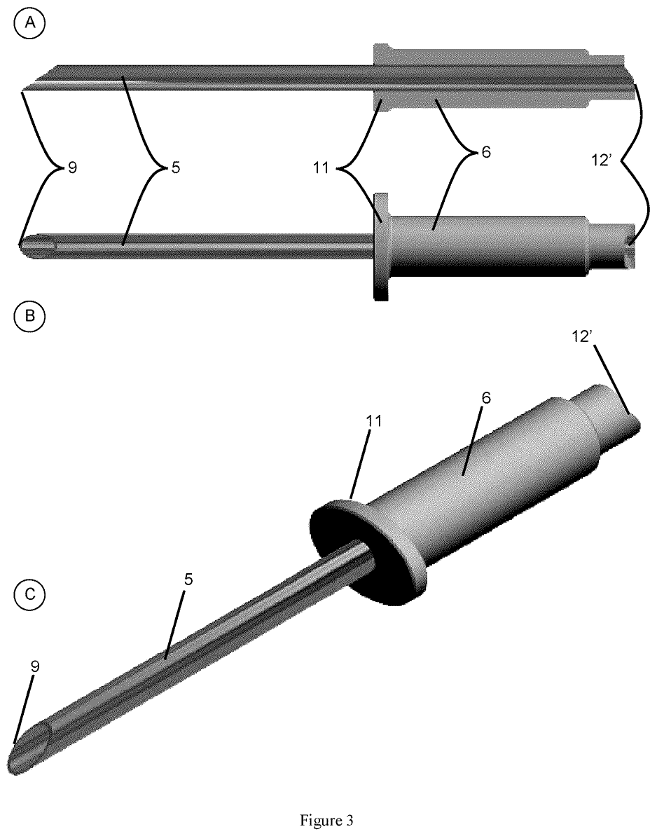

Views A, B, and C of FIG. 3 depict, respectively, a longitudinal cross-section (View A), a top view (View B), and an axonometric view (View C) of the cannulated needle (5), the needle handle (6), and the bevel (9) of the cannulated needle (5). The needle handle (6) encases the cannulated needle (5) and features an oval shape for superior rotation control and includes a hand guard (11) that forms a protective barrier at the front end of the needle handle (6). This hand guard (11) is intended to prevent the surgeon's hand from slipping forward onto the cannulated needle (5) and to provide additional leverage and pushing power during use. The back end surface of the needle handle (6) is equipped with interlocking features (12′) comprising protrusions and recesses. These interlocking features (12′) match opposite-shaped elements on the stylet handle (8), thereby enabling stable locking of the needle handle (6) to the stylet handle (8).

In one embodiment, the needle handle (6) and its interlocking features (12′) are equipped with a sleeve bearing that allows rotational movement of the interlocking features (12′) of the needle handle (6) and those (12″) of the stylet handle (8) locked to the needle handle (6), thereby enabling precise positioning of the stylet handle (8), the stylet (7), the hooked suture thread (1), and its blunt end (3) at any angle relative to the axis of the cannulated needle (5).

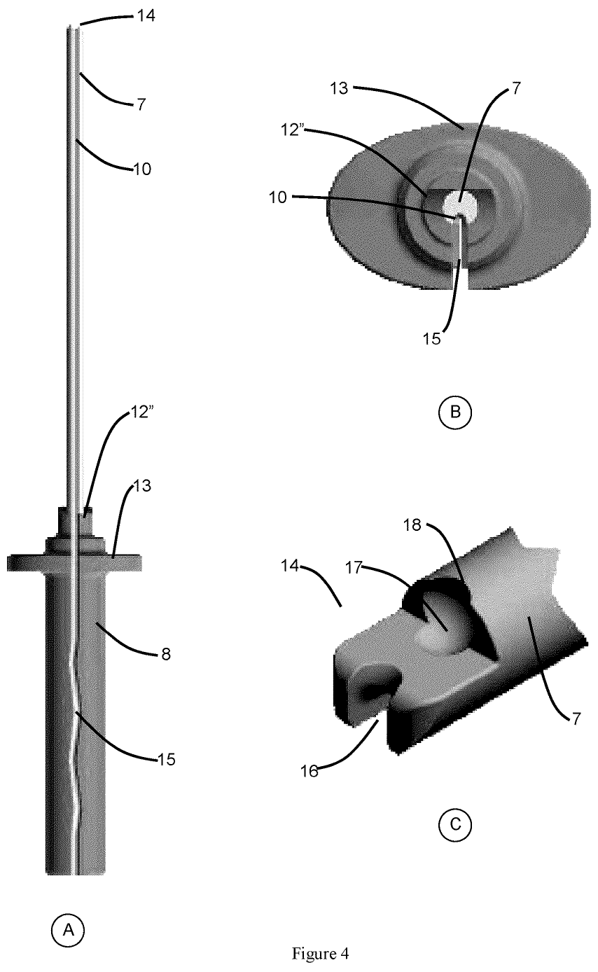

View A of FIG. 4 shows a bottom view of the stylet (7), including the longitudinal channel (10) of the stylet (7), the stylet handle (8) with its interlocking features (12′), a hand guard (13) at the front end of the stylet handle (8), and the tip (14) of the stylet (7). The stylet (7) is a cylindrical element with a diameter that fits within the diameter of the cannulated needle (5), allowing for unobstructed forward, backward, and rotational motion of the stylet (7) within the cannulated needle (5). The length of the stylet (7), excluding its tip (14), corresponds to the combined length of the cannulated needle (5) and the portion encased by the stylet handle (8). The longitudinal channel (10) of the stylet (7) accommodates the suture thread (1) prior to use. The stylet handle (8) encases the stylet (7) and features an oval shape for superior rotation control during use. The hand guard (13) serves as a protective barrier at the front end of the stylet handle (8), preventing the surgeon's hand from slipping forward onto the needle handle (6) and providing additional leverage and pushing power during use. The interlocking features (12″) of the stylet handle (8) are matched and complemental to the protrusions and recesses of the interlocking features (12′) of the needle handle (6). The bottom side of the stylet handle (8) includes a longitudinal channel (15), which facilitates the release of the suture thread (1) from the longitudinal channel (10) of the stylet (7) and from the stylet handle (8) when needed. The longitudinal channel (15) features longitudinal sigmoid curvatures designed to lock and immobilise the suture thread (1) within the longitudinal channel (10) of the stylet (7).

View B of FIG. 4 presents a frontal view of the stylet handle (8), featuring the cross-section of the stylet (7) and its longitudinal channel (10), the interlocking features (12″) of the stylet handle (8), the hand guard (13) of the stylet handle (8), and the cross-section of the longitudinal channel (15) located at the bottom side of the stylet handle (8).

View C of FIG. 4 is an enlarged axonometric view of the tip (14) of the stylet (7). The tip (14) features housings (16) and (17) that are matched and complemental respectively to the hook (2) and the blunt end (3) of the suture thread (1). A retaining cap (18) partially encloses the top of the blunt end (3), functioning to lock the blunt end (3) to its housing (17). The housings (16) and (17) function as tillers, applying forces to the hook (2) and the blunt end (3) of the suture thread (1). This configuration allows the surgeon to rotate the suture thread (1) in conjunction with the stylet (7) and to position the blunt end (3) of the suture thread (1) at any desired angle relative to the axis of the cannulated needle (5).

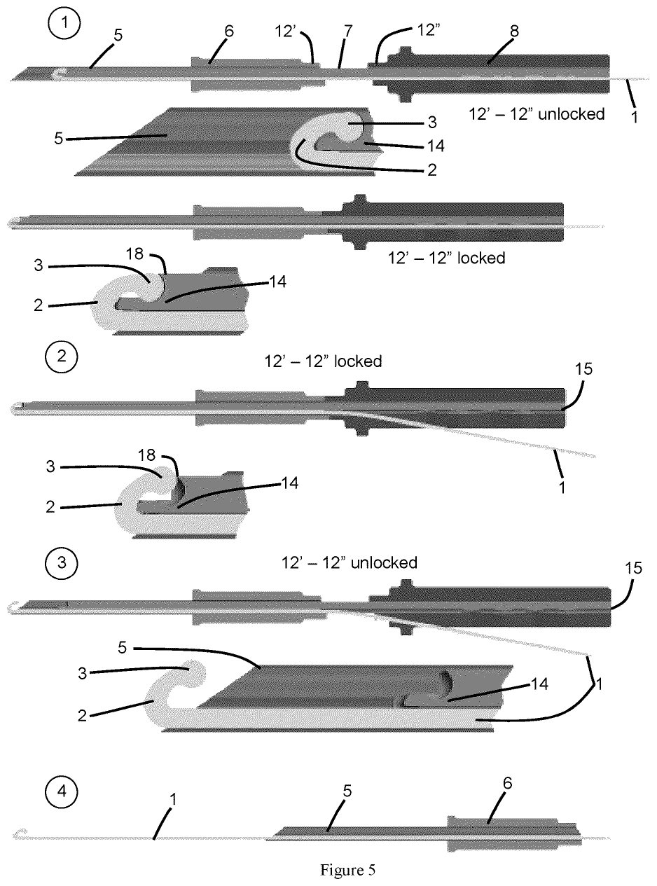

FIG. 5 illustrates the stages of the application of the suture thread (1) by the surgeon, wherein the procedure involves the insertion of the cannulated needle (5) through the skin to access the underlying tissue.

Stage 1 involves the insertion of the suture thread (1) with the assistance of the stylet (7) into the cannulated needle (5), and pushing the suture thread (1) and the stylet (7) towards the tip of the cannulated needle (5), until the hook (2) and the blunt end (3) of the suture thread (1) reach the tip of the cannulated needle (5) and the interlocking features (12′) and (12″) of the needle handle (6) and the stylet handle (8) lock. The surgeon rotates the stylet handle (8), the stylet (7) and its tip (14), the hooked suture thread (1) and its blunt end (3), and positions the blunt end (3) at any desired angle relative to the axis of the cannulated needle (5). At this stage, the blunt end (3) of the suture thread (1) is secured to the tip (14) of the stylet (7) by the retaining cap (18).

At Stage 2, the surgeon unlocks and detaches the hook (2) and blunt end (3) of the suture thread (1) from the tip (14) of the stylet (7) by pulling down and releasing the straight end of the suture thread (1) from the longitudinal channel (10) of the stylet (7) and the longitudinal channel (15) of the stylet handle (8). The surgeon then secures the blunt end (3) of the suture thread (1) to the desired position within the tissue.

At Stage 3, the surgeon retracts the stylet (7) from the cannulated needle (5).

At Stage 4, the procedure concludes with the extraction of the cannulated needle (5) from the tissue and skin. The surgeon finalises the application of the suture thread (1) to the tissue by applying the required tension between the point of application of the blunt end (3) and the point of stitching the straight end of the suture thread (1) to the tissue.

The hook (2) facilitates the easy removal of the suture thread (1) and blunt end (3) when necessary. The removal procedure includes, for example, making a small incision at the dermis near the position of the hook (2) and detaching the straight end of the suture thread (1) from the tissue. This step is followed by interlocking the hook (2) with, for example, a surgical hook retractor, and subsequently pulling the suture thread (1) and the blunt end (3) out.

1. A hooked suture thread with cannulated needle and stylet for precision surgery, comprising:
a suture thread having a first end formed into at least one hook and a second end remaining straight;
at least one aperture along the length of the suture thread;
wherein the hook terminates in a blunt end, the blunt end being positioned and applied at a precise depth and angle within the subdermal or hypodermal layers of skin tissue or other body tissue, and the straight end of the suture thread is pulled to apply pressure to the tissue at the blunt end point, and is stitched through its aperture to another tissue point, thereby creating tension between two distant tissue points.

2. The hooked suture thread with cannulated needle and stylet for precision surgery of claim 1, wherein the suture thread is encased in a longitudinal channel in a stylet, the stylet comprising:
a cylindrical stylet with a diameter that fits within a cannulated needle, allowing for unobstructed forward, backward, and rotational motion within the cannulated needle;
a longitudinal channel accommodating the suture thread;
housings in the stylet tip matched to the hook and blunt end of the suture thread, wherein the housings function as tillers, allowing for the rotation of the suture thread, the hook, and the blunt end, and enabling the positioning of the blunt end at any desired angle;
a retaining cap on the stylet tip to lock the blunt end;
an oval-shaped stylet handle for rotation control and for pushing the stylet and the hooked suture thread through the cannulated needle;
a hand guard on the stylet handle to prevent slipping and provide leverage;
a longitudinal channel in the stylet handle to facilitate suture thread release, featuring sigmoid curvatures to lock and immobilise the suture thread when needed.

3. The cannulated needle for providing a conduit for guiding and positioning the stylet and the hooked suture thread of claims 1 and 2 in the tissue, comprising:
a cylindrical cannulated needle;
a needle handle encasing the cannulated needle;
a bevel on the cannulated needle for tissue penetration;
an oval-shaped needle handle for rotation control;
a hand guard on the needle handle to prevent slipping and provide leverage.

4. The hooked suture thread with cannulated needle and stylet for precision surgery according to any of the preceding claims, wherein the back end surface of the needle handle is equipped with interlocking features comprising protrusions and recesses, the interlocking features matching opposite-shaped elements on the stylet handle, thereby enabling stable locking of the needle handle to the stylet handle.

5. The hooked suture thread with cannulated needle and stylet for precision surgery according to any of the preceding claims, wherein the needle handle and its interlocking features are equipped with a sleeve bearing that allows rotational movement of the interlocking features of the needle handle and those of the stylet handle locked to the needle handle, thereby enabling precise positioning of the stylet handle, the stylet and its tip, the hooked suture thread, and its blunt end at any angle relative to the axis of the cannulated needle.

6. The hooked suture thread with cannulated needle and stylet for precision surgery according to any of the preceding claims, wherein the material of the hook is elastic, allowing for a narrower distance between its blunt end and the suture thread when pressed, and a wider distance when not pressed, with the elasticity and flexibility of the hook material preventing the hook from unbending.

7. The hooked suture thread with cannulated needle and stylet for precision surgery according to any of the preceding claims, wherein the material of the suture thread is of sufficient strength to provide tension between the point on the tissue where the blunt end of the hook is positioned and applied, and the point where the straight end of the suture thread is stitched to the tissue, and said material possesses sufficient flexibility and low friction to allow for unobstructed manoeuvrability within the tissues during placement.

8. The hooked suture thread with cannulated needle and stylet for precision surgery according to any of the preceding claims, wherein the stages of application involve:
inserting the cannulated needle through the skin to access underlying tissue, thereby enabling precise positioning of the blunt end at any desired depth;
inserting the suture thread with the assistance of the stylet into the cannulated needle and pushing the suture thread and stylet towards the tip of the cannulated needle until the hook and blunt end of the suture thread reach the tip of the cannulated needle and the interlocking features of the needle handle and stylet handle lock;
rotating the stylet handle, the stylet and its tip, the hooked suture thread, and its blunt end, enabling precise positioning of the blunt end at any angle relative to the axis of the cannulated needle;
the retaining cap unlocking the hook and blunt end of the suture thread from the tip of the stylet by pulling down and releasing the straight end of the suture thread from the longitudinal channel of the stylet and stylet handle;
securing the blunt end of the suture thread to the desired position within the tissue;
retracting the stylet from the cannulated needle, and extracting the cannulated needle from the tissue and skin;
pulling the straight end of the hooked suture thread to apply pressure to the tissue at the blunt end point;
stitching the straight end through its aperture to another tissue point, thereby creating tension between the two distant tissue points.

9. The hooked suture thread with cannulated needle and stylet for precision surgery according to any of the preceding claims, wherein the hook facilitates the removal of the suture thread and blunt end when necessary, the removal procedure comprising:
making an incision at the dermis near the position of the hook;
dissecting the tissue from the straight end of the suture thread;
interlocking the hook with a surgical hook retractor, and subsequently pulling the suture thread and the blunt end out.
